SEQUENCE LISTING (8 sequences)

SEQ ID NO: 1 - DNA, 33 bp, Artificial Sequence
  Oligonucleotide probe
ntaaccaaagagcaacnacacgaattctcgatn

SEQ ID NO: 2 - DNA, 35 bp, Artificial Sequence
  Oligonucleotide probe
gttgctctttggttaaaaaaaaaaaaaaaaaaaaa

SEQ ID NO: 3 - DNA, 49 bp, Artificial Sequence
  Oligonucleotide probe
ngcgaggcgttttagcgaacctggagtcctggatgcgacgggatctccn

SEQ ID NO: 4 - DNA, 47 bp, Artificial Sequence
  Oligonucleotide probe
ngcgaggcgttttagcgaacccggggcagtgccactgcatcagacan

SEQ ID NO: 5 - DNA, 63 bp, Artificial Sequence
  Oligonucleotide probe
nggttcgctaaaacgcctcgcagggcgacattcaaccgattgggagggcgttcagccctccan

SEQ ID NO: 6 - DNA, 63 bp, Artificial Sequence; identical to SEQ ID NO: 5
  Oligonucleotide probe

SEQ ID NO: 7 - DNA, 66 bp, Artificial Sequence
  Oligonucleotide probe
ngcgattgctcgcagaaaggtctcggtcccgctgtaagttggctaactggtgaggacggtgtctgn

SEQ ID NO: 8 - DNA, 31 bp, Artificial Sequence
  Oligonucleotide probe
naccactcctgccacagacaccaacagaagn

(19) United States
(12) Patent Application Publication
(10) Pub. No.: US 2003/0236205 A1
(43) Pub. Date: Dec. 25, 2003

(54) Hybridization signal amplification method (HSAM) nanostructures for diagnostic and therapeutic uses

(76) Inventors: David Y. Zhang, Jamaica, NY (US); Wandi Zhang, New York, NY (US)

Correspondence Address: Steven B. Pokotilow, Esq., STROOCK & STROOCK & LAVAN LLP, 180 Maiden Lane, New York, NY, 10038, US

(21) Appl. No.: 10/176,515

(22) Filed: Jun. 21, 2002

(51) Int. Cl. A61K 48/00; C12Q 1/68; A61K 39/395

(52) U.S. Cl. 514/44; 424/178.1; 514/8; 435/455; 435/6

(57) ABSTRACT

The present invention relates to a hybridization signal amplification method (HSAM) that can be used to form nanostructures for use in drug delivery and diagnostics and may comprise molecules aimed at a specific target cell of interest. The nanostuctures may be used to treat infectious diseases and physiological disorders such as proliferative, genetic, neurological or metabolic disorders. The nanostructures of the invention comprise nucleic acid molecules having affinity pairs incorporated into their structure. These affinity pairs are formed from ligand and ligand binding moieties that bind to nucleic acid molecules. This bound entity is a complex, web-like structure that serves as a matrix or framework for delivery of therapeutic or diagnostic agents. Since the nanostructures of the invention are comprised of biocompatible and biodegradable materials, such as nucleic acid molecules and proteins, they provide a safe and easily degradable delivery system.

### FIELD OF THE INVENTION

The present invention relates to a hybridization signal amplification method (HSAM) that can be used to form nanostructures for use in drug delivery and diagnostics and may comprise molecules aimed at a specific target cell of interest.

### BACKGROUND OF THE INVENTION

U.S. Pat. No. 5,942,391 describes a general hybridization signal amplification method referred to as HSAM, incorporated herein by reference. In the presence of a target nucleic acid molecule, a web-like complex is formed between target specific probes and signal probes. Detection of the complex indicates the presence of the target nucleic acid. Because of the complex's ability to bind to its complementary sequence thereby facilitating enhanced binding specificity and detection, HSAM is a powerful purification and detection technique.

HSAM is also a novel method enabling the formation of nanostructures, composed of nucleic acid molecules, that are utilized in drug delivery and diagnostic systems. Nanotechnology is a cutting-edge field in which atoms and molecules are engineered to build specific structures for particular purposes. Nanostructures are synthetic molecules that are hundreds of times smaller than the cells of the body—typically ranging in size from 1 to 500 nanometers—and may be composed from a variety of different molecules including those formed using nucleic acid molecules (Seeman et al., 1998, Annu. Rev. Biophys. Biomol. Struc. 27:225-48). Recently, there has been increased interest of nanostructures in biomedical applications, whereby applications include correction of ultrastructural defects in cells, delivery of genes and drugs, and diagnosis of disease. (West and Halas, 2000 Current Opinions in Biotechnology 11:215-217; Hermanson (Ed) Bioconjugate Techniques. San Diego: Academic Press, 1996; Chan et al., 1998 Science 281:2016-2018; and Alvisatos, 2001 Scientific American 67-73).

The need to maximize therapeutic activity while minimizing negative side effects is the driving factor for the development of new drug delivery systems (Rosler et al., 2001 Advanced Drug Delivery Rev. 53: 95-108). When discussing the effectiveness of a drug therapy, two important issues are taken into consideration—the extent to which temporal control and distribution control can be achieved. Temporal control refers to the ability to adjust the period of time during which drug release is supposed to take place; it also refers to the triggering of the release process at a specific time during treatment. Distribution control relates to directing the drug delivery system precisely to the desired site of activity.

The present invention provides novel methods and compositions comprising nucleic acid-based nanostructures. HSAM can be used to assemble a nanostructure in a bottom-up scheme, in that the assembly takes advantage of some of the unique structural capabilities of nucleic acids and ligand-binding molecules.

Applications of HSAM are, however, very broad and not limited to nanotechnology. HSAM can be used to improve detection of a target nucleic acid vis-à-vis an enhanced signal and a physically large complex that can be formed and visualized with the naked eye. Additionally, in the presence of certain specific instruments (e.g., luminx), HSAM can improve assay sensitivity to the single-molecule level, thereby eliminating the need for pre-amplification of target nucleic acids. Alternatively, HSAM can be used in a DNA assay format. In this case, the signal can be improved to detect lower copy numbers of target nucleic acids. Finally, HSAM can be used to facilitate the capture of target nucleic acids. HSAM can aggregate paramagnetic particles via a capture probe and can be used to capture specific target DNA or simply to extract total mRNA. The resultant, purified mRNA can then be used directly for RT-PCR or RAM amplification (U.S. Pat. No. 5,942,391).

### SUMMARY OF THE INVENTION

The present invention relates to a hybridization signal amplification method (HSAM) that can be used to form nanostructures for use in drug delivery and diagnostics and may comprise molecules aimed at a specific target cell of interest. The nanostructures of the invention comprise nucleic acid molecules having ligand and/or ligand binding moieties incorporated into their structure. These ligand and ligand binding moieties form an affinity pair that binds to nucleic acid molecules. This bound entity is a complex, web-like structure that serves as a matrix or framework for delivery of therapeutic or diagnostic agents.

The size and shape of the nanostructures may be controlled in a variety of different ways. For example, the size of the nucleic acid molecules can be altered. Additionally, the type and concentration of ligand and/or ligand binding moieties may be modified.

The nanostuctures of the invention may be used to treat infectious diseases and a variety of different physiological disorders, such as proliferative, genetic, neurological or metabolic disorders. The nanostructures may also be used in immunization protocols to introduce nucleic acids encoding immunogens, or the immunogens themselves, into the subject to stimulate a protective immune response. The methods and compositions of the invention also provide a means for bioimaging (i.e., delivery of diagnostic agents, such as fluorescent or chemiluminescent agents) to ascertain the diagnosis and/or prognosis of disease in a subject.

The present invention provides a drug delivery system that can transport specific molecules (e.g., proteins, peptides, chemical compounds) to affected sites, as needed. The introduction of proteins and peptides, to treat conditions such as anemia, growth hormone deficiency, diabetes and multiple sclerosis, has led to immense therapeutic and quality-of-life benefits for patients. However, the fragile nature of proteins and peptides virtually eliminates the possibility of oral administration; in oral form, proteins and peptides are quickly degraded in the stomach via enzymes and hydrochloric acid. Alternatively, transdermal delivery has met with limited success given issues of size and inherent physical properties that make it difficult for proteins and peptides to cross skin layers, as well as mucosal (nasal) delivery. A drug delivery system that can convey a protein or peptide to an affected site would thus have benefical therapeutic consequences for patients.

The present invention also provides a drug delivery system that enables a clinician to locally infuse a therapeutically high concentration of toxic agent(s) without adversely affecting the outlying areas. The use of toxic agents in the treatment of many disorders is well accepted, but not without negative consequence to healthy or unaffected surrounding tissue. Toxicity to healthy tissue is often dose-limiting, a fact that can adversely affect treatment of the disease and result in a negative outcome for the patient. A drug delivery system that can achieve a localized infusion of agent at a high enough dosage to treat the disease without resulting in systemic toxicity would enhance the therapeutic effects of the treatment.

The methods and compositions of the invention further provide a means for delivery of bioimaging agents (e.g., fluorescent agents, bioluminescent agents, chemiluminescent radioactive agents) into a test subject for diagnostic purposes. The nanostructures may be designed to identify target cells expressing specific genes known to be associated with a particular disorder or disease. By including nucleic acid molecules that are complementary and in anti-sense orientation to the specific gene into the nanostructure, the complex can be aimed at and thereby, directly affect, a specific target cell. Once the subject has been exposed to the nanostructures, the presence of the bioimaging agent can be detected using a number of methods well known to those of skill in the art.

One of the advantages of HSAM nanoparticles is that sophisticated instrumentation and chemistry is not required. Rather, the HSAM nanostructures can be formed using simple building blocks (i.e., DNA, avidin). Furthermore, the HSAM nanostructures' formation is self-assembling and results from simple building blocks in a bottom-up format. A ligand-derivatized nucleic acid can self-assemble into a structurally defined nanostructure, comprised of short nucleic acids and multivalent ligand-binding molecules. This can be a great advantage in certain assays in which the small, initial molecules can be assembled into large molecules that can facilitate the detection of signal. Molecular size and shape can be controlled by modifying the length of the HSAM probe, the amount of biotin, and the ratio of biotin to avidin.

### BRIEF DESCRIPTION OF THE DRAWINGS

FIG. 1 is a schematic diagram of three possible HSAM nanostructures formed via affinity pair binding. A signal probe containing three biotin molecules (round circles) is used to form a complex with avidin (crosses). The numbers indicate the number of nucleic acid strands held by each avidin molecule.

FIG. 2 is a schematic diagram of the formation of a HSAM nanostructure through signal probe hybridization. Five signal probes are used: A, B, C, D, and Y. A′, B′, C′, D′, and Y′ designate probes with complementary sequences, respectively.

FIG. 3 is a schematic diagram of target sequence directed formation of a HSAM nanostructure. There are two target sequences (Target A and Target B) that are bound to target sequence specific signal probes (A′ and B′). Probes A′ and B′, which contain avidin molecules, are then bound by additional signal probes containing biotin molecules (circles).

FIG. 4 is a schematic diagram illustrating the use of differentially labeled signal probes to generate a signal once the two probes are co-localized on a target sequence.

FIG. 5 depicts the visualization of HSAM nanostructures following staining with blue dye. HSAM nanostructures were formed in the presence of avidin and the HSAM signal probe and then stained with a blue dye. (A) Image was taken at 50× magnification. (B) Image was taken at 600× magnification. (C) Schematic representation of HSAM nanostructure where the arrows represent biotin molecules incorporated into the signal probe and the crosses represent avidin molecules.

FIG. 6 depicts the sequence specific binding of magnetic particles to HSAM nanostructures. In the presence of signal probe #1, containing the poly-dA sequence at the 3′ end and the complementary sequence to a portion of probe #2 at the 5′ end, the poly-dT coated magnetic particle was able to bind to the HSAM nanostructure that was formed by binding biotinylated (arrow) signal probe #2 to avidin (cross). The results are visualized in panels A, B, C, and D. Panels A and B depict binding in the presence of signal probe #1 at 100× and 400× magnification, respectively. Panels C and D depict binding in the absence of signal probe #1 at 100× and 400× magnification, respectively.

FIG. 7A depicts the microscopic visualization (100× magnification) of the formation of HSAM nanostructures using signal probe hybridization. FIG. 7B is a schematic representation of this type of nanostructure.

FIG. 8 depicts the detection of target DNA nucleic acid sequences using HSAM nanostructures via dot blot. Panel A, depicting the control experiment, shows a dot blot with decreasing numbers of target DNA nucleic acid molecules from left to right on the blot (i.e., 10¹² down to 10⁰ molecules). Panel B is a schematic diagram of the biotinylated (arrow) target DNA nucleic acid molecule bound to avidin (cross). No HSAM nanostructures have been added to the sample. Panel C, depicting the HSAM nanostructure experiment, shows a dot blot with increasing numbers of target DNA nucleic acid molecules from left to right on the blot (i.e., 10⁰ up to 10⁸ molecules). HSAM nanostructures were added to the sample thereby resulting in complex formation as depicted schematically in Panel D.

FIG. 9 depicts the visualization of doxorubicin binding to HSAM nanostructures. The Dox panels (C and D) depict binding of doxorubicin to the HSAM nanostructures. The Control panels (A and B) do not contain doxorubicin.

FIG. 10 is a graphical representation of the binding capacity of doxorubicin and HSAM nanostrucutures. The control sample did not contain any HSAM nanostructures and the HSAM sample contained both doxorubicin and HSAM nanostructures. The absorbance measurements are indicated on the Y-axis.

FIG. 11 depicts the inhibition of cancer cell growth by HSAM-DOX nanostructures. Panel A shows the HSAM-DOX spotting area and cell growth inhibition is demonstrated. Panel B shows an area distal to the HSAM-DOX spotting area and no cell growth inhibition is demonstrated.

### DETAILED DESCRIPTION OF THE INVENTION

The present invention relates to HSAM that can be used to form novel nanostructures for use in drug delivery and diagnostics (both in vitro and in vivo). The nanostructures of the invention comprise nucleic acid molecules bound to one another to form a complex, or web-like structure, that serves as a matrix or framework for delivery of therapeutic or diagnostic agents. Since the nanostructures of the invention are comprised of biocompatible and biodegradable materials, such as nucleic acid molecules and proteins, they provide a safe and easily degradable delivery system.

The nanostuctures may be used to treat infectious diseases and physiological disorders such as proliferative, genetic, neurological or metabolic disorders. The nanostructures may also be used in immunization protocols where the desire is to introduce nucleic acids encoding immunogens, or the immunogens themselves, into the subject to stimulate a protective immune response. In addition, the methods and compositions of the invention provide a means for bioimaging, (i.e., delivery of diagnostic agents to a targeted cell or tissue), for the detection of disease in a test subject.

The nanostructures of the invention comprise nucleic acid molecules, referred to herein as signal probes, which are bound to one another to form a branched complex that serves as a matrix or framework for delivery of therapeutic or diagnostic agents. The nanostructures are formed by contacting a mixture of nucleic acid molecules having affinity pairs incorporated into their structure. In one embodiment of the invention, the affinity pair consists of a “ligand” and “ligand binding moieties.” The term “ligand” as used herein refers to any component that has an affinity for another component termed here as “ligand binding moiety.” Upon binding of the “ligand” and/or “ligand binding moieties,” an affinity pair is formed. Such affinity pairs include, but are not limited to, biotin with streptavidin/avidin, antigens or haptens with antibodies, heavy metal groups with thiogroups, polynucleotides such as homopolynucleotides (e.g., poly dG with poly dC, poly dA with poly dT and poly dA with poly dU), lectins with glycoproteins, ligands with receptors, proteins with proteins, DNA with DNA, RNA with DNA, RNA with RNA, and chemical activators with receptors. In a further embodiment the affinity pair consists of multiple polynucleotides whose sequences are complementary to one another. Upon complementary hybridization the affinity pair is formed.

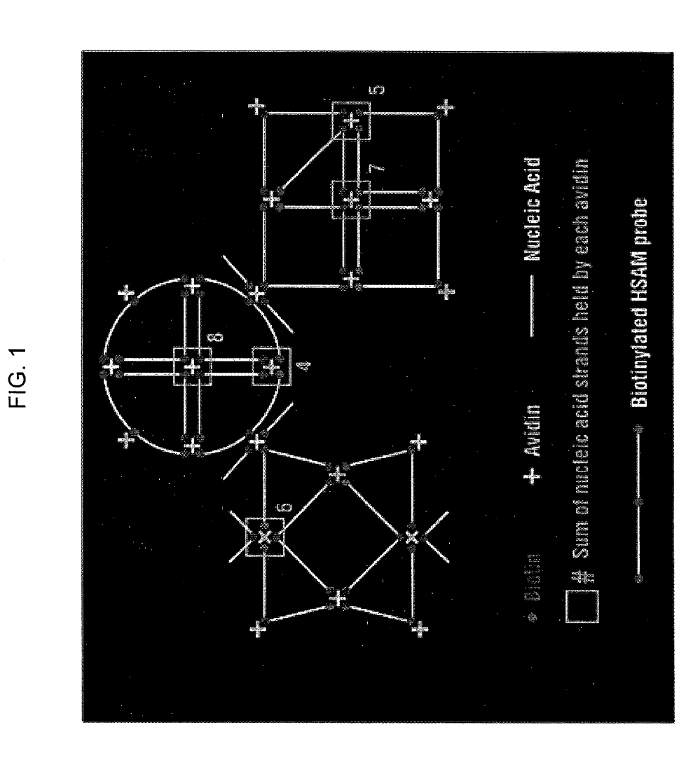

In some instances, it may be necessary to add a ligand or ligand binding moiety to the reaction to form a bridge between the complexed nucleic acid molecules. A matrix-like complex is formed as depicted schematically in FIG. 1.

While not wishing to be bound by any one theory, the principle of formation of HSAM nanostructures is that interactions between components of the affinity pairs, will cause the nucleic acid molecules to bind to one another to form a web-like complex. For example, binding of avidin to biotin moieties within the nucleic acid molecules will result in formation of a complex between the nucleic acid molecules.

Thus, to form the nanostructures of the invention, the nucleic acid molecules (i.e., signal probes), are contacted with one another under conditions that permit binding of the ligand to the ligand binding moiety. The conditions to be used will depend on the ligand and ligand binding moiety used in the signal probe. Such conditions are well known to those of skill in the art.

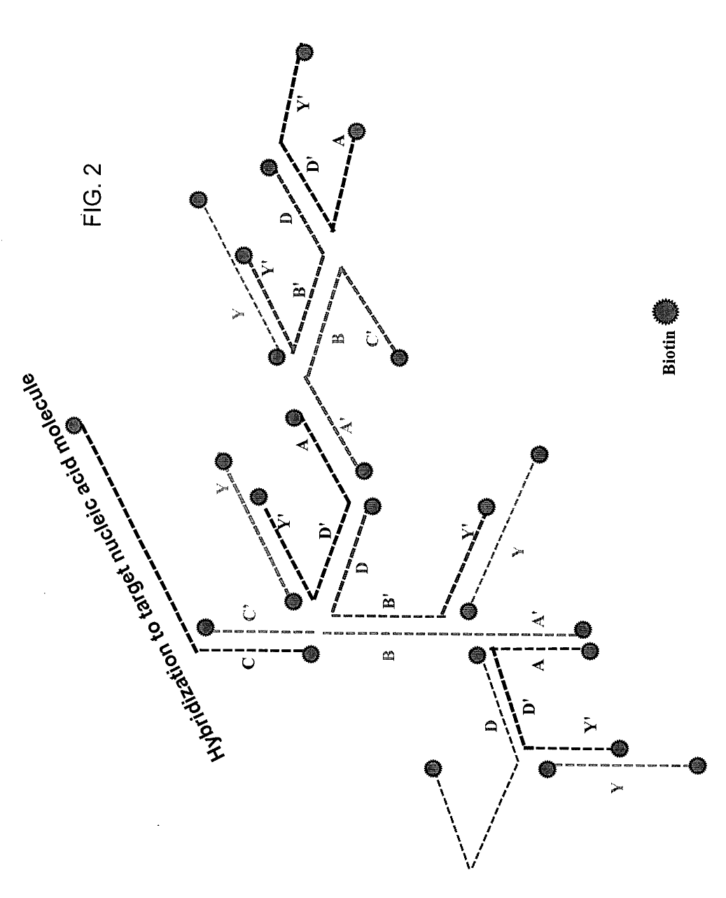

In a further embodiment, the nucleic acid molecules may be designed to have complementary sequences at their 5′ and/or 3′ ends, thereby permitting binding of the nucleic acid molecules to one another through their complementary ends. The binding affinity between the nucleic acid molecules will result in the formation of a complex of nucleic acid molecules as depicted schematically in FIG. 2. In this embodiment the presence of the affinity pair will serve as a detection mechanism. This will be particularly useful in diagnostic applications where the initial binding step will be the sequence specific HSAM signal probe binding to a target nucleic acid. Additional signal probes are added and the nanostructure will self-assemble through complementary hybridization.

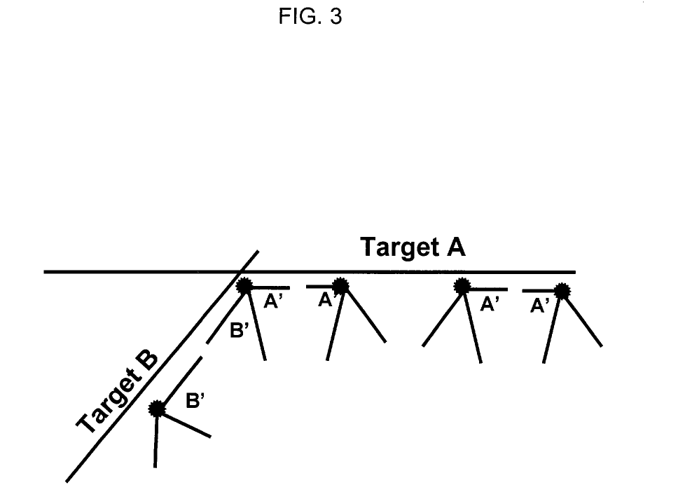

In another embodiment of the invention, two signal probes can be designed as depicted in FIG. 3. One signal probe is complementary to the target sequences (e.g., Target A or Target B). The second signal probe does not bind to the target sequences but contains ligand binding moieties thereby permitting formation of branched complexes. The signal probes may be designed to bind to target sequences at a variety of different locations. Binding of the branched complex to the first signal probe co-localizes the detectable HSAM nanostructure to the target nucleic acid.

Receptor-mediated transfer may involve linking the nucleic acid molecules to a polycationic protein (usually poly-L-lysine) containing a covalently attached ligand. This complex is selected to target a specific receptor on the surface of the cell of interest. The nanostructure is then taken up by the cell, thereby directly targeting the therapeutic or diagnostic agent carried by the nanoparticle. Cell-specific delivery of a nanostructure using a conjugate of a polynucleic acid binding agent (e.g., polylysine, polyarginine, polyornithine, histone, avidin, or protamine) and a tissue receptor-specific protein ligand may be achieved using the method of Wu et al. (U.S. Pat. No. 5,166,320).

Nucleic acids useful in the present invention may be prepared by any conventional means typically used to prepare nucleic acid molecules in large quantities. For example, DNA may be chemically synthesized using commercially available reagents and synthesizers by methods that are well-known in the art (Gait, 1985, Oligonucleotide Synthesis: A Practical Approach, IRL Press, Oxford, England). The nucleic acid molecules can be modified at the base moiety, sugar moiety, or phosphate backbone to improve stability of the molecule, hybridization, transport into the cell, and/or increase the binding capacity of a drug. Modification of a nucleic acid molecules to reduce the overall charge can enhance the cellular uptake of the molecule. In addition, modifications can be made to reduce susceptibility to nuclease degradation.

The nucleic acid molecules may include other appended groups such as peptides (e.g., for targeting host cell receptors in vivo), or agents facilitating transport across the cell membrane such as phospholipids (Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (PCT Publication No. W089/10134, published Apr. 25, 1988).

Various, other, well-known modifications to the nucleic acid molecules can be introduced as a means of increasing intracellular stability, half-life and/or to control the rate of release of agents from the nanoparticles. Possible modifications include, but are not limited to, the addition of flanking sequences of ribo- or deoxy-nucleotides to the 5′ and/or 3′ ends of the molecule. In some circumstances where increased stability is desired, nucleic acids having modified internucleoside linkages, such as 2′-0-methylation, may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein). Nucleic acid/peptide hybrid molecules that are more resistant to nuclease degradation and have an enhanced affinity for a target nucleic acid molecule may also be utilized (Kushon S A et al., 2001, J. Am Chem Soc. 2001 123:10805-13).

The nucleic acids may be purified by any suitable means, as are well known in the art. For example, the nucleic acids can be purified by reverse phase chromatography or gel electrophoresis. The skilled artisan will recognize that the method of purification will depend in part on the size and charge of the nucleic acid to be purified.

The size and shape of the nanostructure may be precisely modulated by using nucleic acid molecules of different sizes and nucleotide sequences. Thus, the use of shorter nucleic acid molecules may result in the formation of a nanostructure with a more compact structure (i.e., a smaller mesh size). Alternatively, the use of longer nucleic acid molecules may result in the formation of a nanostructure with a less compact structure (i.e., larger mesh size).

In a specific embodiment of the invention, the nucleic acid molecules are 10-1000 bases long. In a further embodiment of the invention, the nucleic acid molecules are 25-100 bases long. In some instances, it may be desirable to utilize nucleic acid molecules with a predicted secondary structure. Such secondary structures may be advantageously used to introduce different nucleic acid molecule morphology into the nanostructures. In addition, the type and concentration of the binding moieties incorporated into the signal probes may be selected based on their ability to modulate the size and shape of the nanostructures.

The size of the nanostructure can also be modulated as the result of multiple valency of the ligand binding molecule. When the ligand in the target specific signal probe is biotin, one molecule of streptavidin binds per molecule of biotin in the probe. The bound streptavidin is capable of binding to three additional molecules of biotin. When the non-target specific signal probe is added, the biotin molecules on the signal probe bind to the available binding sites of the streptavidin bound to the target specific signal probe.

The desired size and shape of the nanostructures will depend on the therapeutic/diagnostic agent to be delivered by the nanostructure. The agents to be delivered can be either bound directly to the nucleic acid molecules or may be packaged within the core nanostructures. The nanostructures of the invention provide a means for delivering therapeutic agents to those subjects having infectious diseases or physiological disorders, including proliferative disorders, in which treatment with a specific agent is known to alleviate the symptoms of the particular disease or disorder. By way of examples, the nanostructures may be designed to deliver chemotherapeutic agents, chemosensitizers or radiosensitizers to a subject suffering from a proliferative disorder such as cancer. In addition, the nanostructures of the invention may be engineered to deliver agents useful for treatment of neurological, metabolic or genetic disorders. For treatment of a metabolic disorder such as diabetes, insulin may be packaged into the nanostructures in such a way as to promote controlled release of the protein into the subject. For treatment of infectious diseases, the nanostructures may be designed to deliver therapeutic agents known to kill or inhibit the growth of the infectious microorganism, (i.e., bacteria, virus, parasite of fungi). Such agents include, but are not limited to, antibiotics, viral inhibitors and anti-parasitic agents. The agents to be incorporated into the nanostructures will depend on the disorder to be treated and are known to those of skill in the art.

When choosing the affinity pair to be used it may be advantageous to select moieties that may also serve to target the nanostructure to the target cell of interest. Specific ligands, such as growth factors, hormones, or antibodies may be selected to target the nanostructures to a specific receptor or protein expressed on the surface of the target cell. The nanostructure is then taken up by the target cell and efficiently delivers the drug/or bioimaging agent to the target cell or tissue of interest. In a specific embodiment of the invention, the nanostructures of the invention can be used for the delivery of agents to a specific target site, thus achieving low toxicity and/or controlled release of the agent over prolonged periods of time.

The present invention also provides for nucleic acid molecules used in conjunction with a biocompatible matrix to produce the nanostructures of the invention. The type of matrix that may be used in the compositions and methods of the invention is virtually limitless, as long as it is a “biocompatible matrix.” This means that the matrix has all the features commonly associated with “biocompatibility” in that it is in a form that does not produce an adverse, allergic or untoward reaction when administered to a subject (i.e., an animal, human, mammal, or vertebrate). The biocompatible matrix is also suitable to be placed in contact with cells or tissue. The matrices provide an increased surface or reaction area for delivery of therapeutic or diagnostic agents and a means for controlling the time release of such agents. Such biocompatible matrices include, but are not limited to, hydrogels, agarose, acrylamide, dextran or any other pharmaceutically acceptable carrier.

Applicants note that it is not required that a biocompatible matrix be utilized in conjunction with the claimed invention. In the event that a biocompatible matrix is not used, the nanostructure would be administered directly to the desired site of action. One such means of direct administration is injection, either subcutaneously, transcutaneously or following surgical exposure of the affected site(s).

The present invention also provides for compositions comprising an effective amount of a nanostructure and an acceptable carrier. In a further embodiment, the acceptable carrier is a pharmaceutically acceptable carrier. In a specific embodiment, the term “pharmaceutically acceptable” means and entity that has been approved by a regulatory agency of the Federal or state government, or listed in the U.S. Pharmacopeia or other generally recognized pharmacopeia for use in animals and humans. The term “carrier” refers to a diluent, adjuvant, excipient, or vehicle with which the nanostructure is administered. Examples of suitable pharmaceutical carriers are described in “Remington's Pharmaceutical Sciences” by E. W. Martin.

In a specific embodiment of the invention, the therapeutic or diagnostic agent of interest may be incorporated into a matrix material such as agarose or a hydrogel material. Addition of nucleic acid molecules (i.e., HSAM signal probes) will result in encapsulation of the matrix material within the HSAM nanostructure. Optionally, the agarose or hydrogel material may be composed of a temperature sensitive material that can be removed from the nanostructures by heating (i.e., melting, leaving the therapeutic or diagnostic agent encapsulated within the nanostructure). In one aspect of the invention, the nanostructures are formulated into pharmaceutical compositions designed to allow for sustained release of the drug carried by the nanostructure over prolonged periods of time. Any biocompatible matrix material, such as a hydrogel, containing a nanostructure can be formulated and used in accordance with the invention.

Alternatively, the therapeutic or diagnostic agents may be intercalated within the nucleic acid molecules (i.e., HSAM signal probes) prior to formation of the nanostructures. Upon entry into the target cell, nuclease digestion of the nucleic acid molecules results in release of the therapeutic or diagnostic agent within the cell. In a specific embodiment of the invention, the double stranded nucleic acid molecules may be engineered to contain nuclease recognition sites to facilitate digestion of the nanostructures once inside the cell.

The delivery methods and compositions of the present invention will have a wide range of applications as a drug delivery method for treating infectious diseases, proliferative disorders, genetic disorders, neurological orders or metabolic disorders. In a specific embodiment of the invention, the nanoparticles may be designed to deliver highly toxic chemotherapeutic agents to cancer cells. Anticancer drugs, such as doxorubicin, can be intercalated into nucleic acid molecules. When administered to the subject, the agent is initially inactive because the drug is bound to the nucleic acid molecule. However, once taken up by the tumor cell or tumor tissue, nucleases endogenous to the tumor cells or released by tumor tissue, will degrade the nanostructures (i.e., DNA) thereby releasing the agent into the cell. Using such methods, the therapeutic agent may be delivered in a concentrated form directly to the tumor cell, where it can achieve an antiproliferative effect as well as diminish the negative systemic effects of the agent. Additionally, the nanostructures may be designed to deliver agents capable of sensitizing cells to radiation, or other forms of chemotherapy. For example, nanostructures can be designed to deliver metals to target cancer cells, thereby heightening the cells' sensitivity to radiation.

In another embodiment of the invention, the nanostructures may be used in gene therapy, in which the desired goal is delivery of a therapeutic nucleic acid molecule into a target cell. The nucleic acid molecules may encode for a protein or may function to prevent the expression of a target gene (i.e., antisense, RNA interference (RNAi) molecules and ribozyme molecules). The nanostructures may encode for the therapeutic nucleic acid molecule, or, alternatively, the therapeutic nucleic acid molecule may be packaged within the nanostructure, thereby protecting the gene from degradation.

In a specific embodiment of the invention, a gene encoding, for example, thymidine kinase, can be mixed with a biocompatible matrix to form a core structure that can be enclosed within the shell of the nanostructure. The nanostructures can be further linked to a ligand binding moiety that is capable of targeting to a target cell (i.e., a tumor cell).

In yet another embodiment of the invention, the delivery methods and compositions of the present invention can be used to immunize a subject with an antigen to elicit an immune response. Nanostructures can be designed to deliver a protein antigen, or to deliver a nucleic acid molecule capable of encoding a protein antigen, into the subject to be immunized. In a specific embodiment of the invention, the signal probes that form the nanostructure may encode the protein antigen of interest. For administration, the nanostructures may be formulated with a suitable adjuvant in order to enhance the immunological response to the protein antigen.

In yet another embodiment, the methods and compositions of the invention provide a means for delivery of bioimaging agents into a subject for diagnostic uses. The nanostructures may be designed to identify target cells expressing specific genes, known to be associated with a particular disorder or disease by including nucleic acid molecules in the structure of the nanostructure. These nucleic acid molecules are complementary to and in anti-sense orientation to the specific gene.

Bioimaging agents that may be used in the practice of the invention include but are not limited to, fluorescent, bioluminescent, chemiluminescent or radioactive agents. Bioluminescent molecules include, but are not limited to, firefly, Renilla or bacterial luciferase. Fluorescent molecules include, but are not limited to, green fluorescent protein or red fluorescent protein. In addition, metals such as gold or silver, which have the ability to scatter light may be packaged into nanostructures. Once the subject has been exposed to the nanostructures, the presence of the bioimaging agent can be detected using a number of methods well known to those of skill in the art. Such methods include microscopy, use of a CCD low-light monitoring system, positron emission tomography (PET), single photon emission computed tomography (SPECT), magnetic resonance imaging (MRI), ultrasound (US), and endoscopic optical coherence tomography.

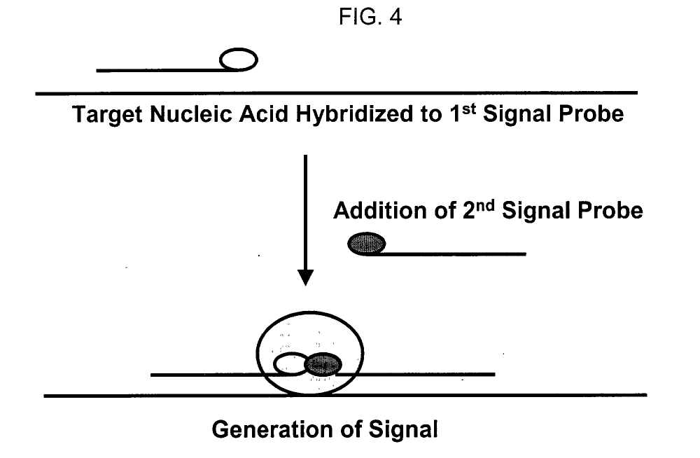

In a further embodiment, two signal probes may be used that are labeled with chemiluminescent or fluorescent moieties. The fluorescent or chemiluminescent moieties may be bound to the signal probe via the ligands incorporated into the probe or, alternatively, bound to the terminus of signal probe. Such signal probes, which are capable of forming a HSAM nanostructure, are used in conjunction with a target specific signal probe to co-localize the HSAM nanostructure to the target nucleic acid. The labeling moieties to be used are chosen based on their ability to transmit a specific signal when both the target specific signal probes are bound to the target nucleic acid molecule, thereby positioning the two HSAM signal probes in close proximity to one another. The energy generated from one of the HSAM nanostructure is capable of transfering energy to the other bound HSAM nanostructure, leading to generation of a signal. This is depicted schematically in FIG. 4.

The present methods may be used with routine clinical samples obtained for testing purposes by a clinical diagnostic laboratory. Clinical samples that may be used in the present methods include, inter alia, whole blood, separated white blood cells, sputum, urine, tissue biopsies, throat swabbings and the like (i.e., any patient sample normally sent to a clinical laboratory for analysis).

### EXAMPLES

### Example 1

Visualization of HSAM Nanostructures Using Biotinvlated Signal Probes and Avidin

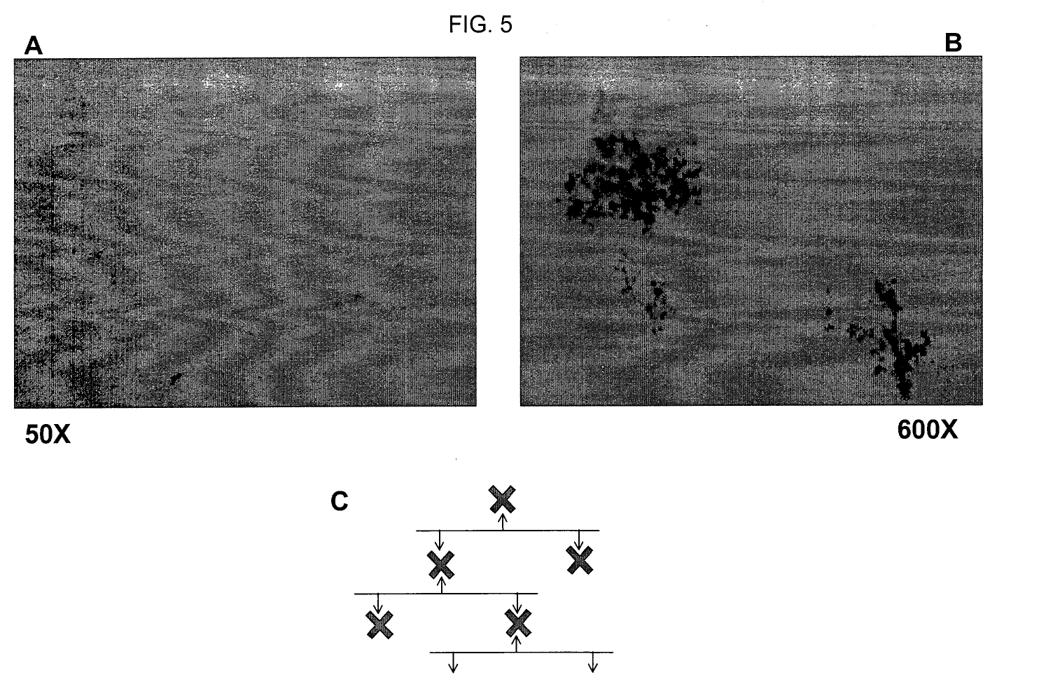

Into an eppendorf tube, 100 mM Tris-HCl buffer (pH 7.5) containing 150 mM NaCl, 0.5-12 μL of 0.4 mg/ml avidin (Pierce), and 0.5-8 μL of 59.5 ng/ml signal probe (SEQ ID No.1) labeled with three biotin molecules were added to a final total volume of 20 μL. This resulted in an avidin:biotinylated DNA ratio of 3:4. The tubes were vortexed and incubated at room temperature for 30 minutes with rocking. 10 μl of methylene blue dye (Richard-Allen Scientific) was added to the tube and incubated at 25° C. for 5 min. A 10 μl aliquot was transferred onto a glass slide and covered with a cover slip. No HSAM nanostructures could be seen under the same magnification if the mixture was made by the ratio of 3:40 or 30:4 (data not shown). The size of HSAM nanostructures formed under the above conditions ranges from 10 to 100 micrometers which can be easily observed under as depicted in FIG. 5. (A) 50× magnification; (B) 600× magnification; (C) schematic presentation of signal probe-avidin nanostructure.

### Example 2

Attraction of Magnetic Particles to HSAM Nanostructures through Signal Probes

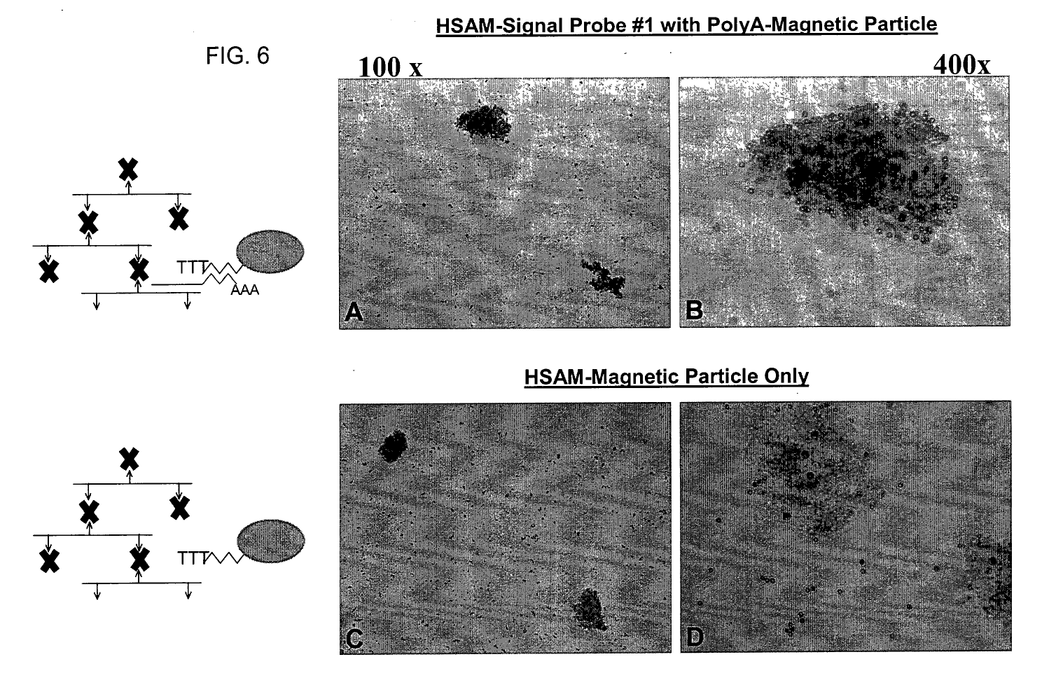

10¹⁴ molecules of signal probe #1 (SEQ ID No. 2), containing a poly-dA at its 3′ end and a sequence complementary to signal probe #2 (SEQ ID No. 1) at its 5′ end, were incubated with the HSAM nanostructures derived from 4×10¹³ molecules of biotinylated HSAM signal probe #2 and 3×10¹³ molecules of avidin. After 10 minutes of incubation at 37° C., the mixture was centrifuged at 8,000 rpm for 3 minutes. The pellet was re-suspended in 10 μl of Tris buffer (same as in Example 1) and incubated with 2 μl of magnetic particles (10⁶ molecules), coated with poly-T₂₅, pre-balanced in the same Tris buffer at 37° C. for 1 hour with shaking. The mixture was stained with 1 μl of methylene blue dye (Richard-Allen Scientific) and checked under a microscope. As a control, the HSAM nanostructure was incubated with the poly-T₂₅ magnetic particles in the absence of signal probe #1. The resulting sequence specific attraction of the magnetic particles to the nanostructures is depicted in FIG. 6. The results showed that a large number of magnetic particles bind to the HSAM nanostructure in the presence of signal probe #1 as shown in Panels A and B. In the absence of signal probe #1, the magnetic particles are scattered in the background as shown in Panels C and D.

### Example 3

Formation of Nanostructures Using Signal Probe Hybridization

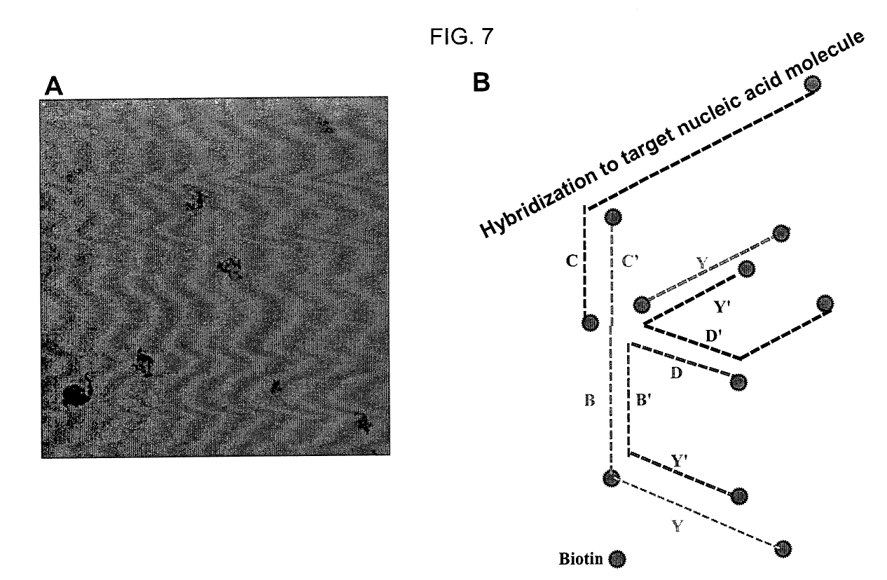

1 μl of each signal probe (SEQ ID No. 3, 4, 5, 6 and 7) at 10¹³ molecules/μl concentration was added to 25 μl H₂O in a 0.5 ml eppendorf tube and incubated at 52° C. for 30 min. The signal probes have sequences that are complementary to each other and therefore facilitate the formation of the nanostructure. 1 μl of avidin (1.3×10¹³ molecules) was added to the tube and incubated at 25° C. for 10 min. 10 μl of methylene blue dye (Richard-Allan Scientific) was added to the tube and incubated at 25° C. for 5 min. A 15 μl aliquot was transferred onto a glass slide and covered by a cover glass. The stained nanostructures were observed under the microscope at 100× magnification. The microscopic results are depicted in FIG. 7A and a schematic representation of this type of nanostructure is depicted in 7B.

### Example 4

Dot Blot Detection of Nucleic Acid with HSAM Nanostructures

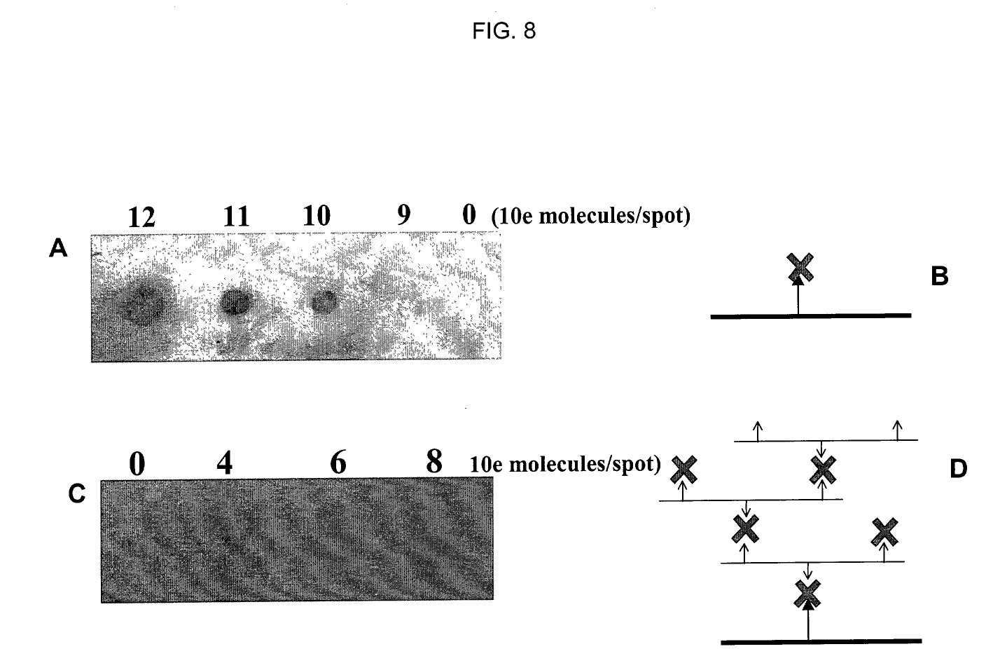

5 μl of biotinylated target DNA (SEQ ID No. 8) was dotted onto a strip of Hybond N+ Nylon membrane (USB) and the DNA was immobilized with UV irradiation for 3 min. The membrane was then soaked in 0.84 ml 1× PBS, 100 μL 50× Denhart's and 60 μl of 10 mg/ml salmon sheared DNA at 45° C. for 30 min on a rotor to block the membrane. In the experimental sample, the HSAM signal probe (with 3 biotins) (SEQ ID No. 1) was added to a final concentration of 3.7×10¹² molecules/ml (300 ng/ml) and incubated for 15 min at room temperature. In the controal sample the HSAM signal probes were not added. Streptavidin was added to a final concentration of 10¹³ molecules/ml (1.2 μg/ml) and biotinylated horseradish peroxidase (HRP)(Pierce) was added to a final concentration of 1 μg/ml and incubated at room temperature for 20 min on a shaker. The membrane was washed 4 times with 14 ml TBSBT buffer (25 mM Tris-HCL, 150 mM NaCL, 0.1% Tween-80,1% BSA, pH 7.5)(Pierce) without shaking. The membrane was placed on 3MM paper and allowed to dry. 0.5 ml of a SuperSignal West Pico Chemiluminescent substrate (Pierce) working solution was added at room temperature for 3 min followed by drying of the membrane. The membrane was then exposed to Kodak film for 1-20 minutes. FIG. 8 depicts the detection of biotinylated target DNA with HSAM nanostructures (Panels C and D).

### Example 5

Binding of Doxorubicin to HSAM Nanostructures

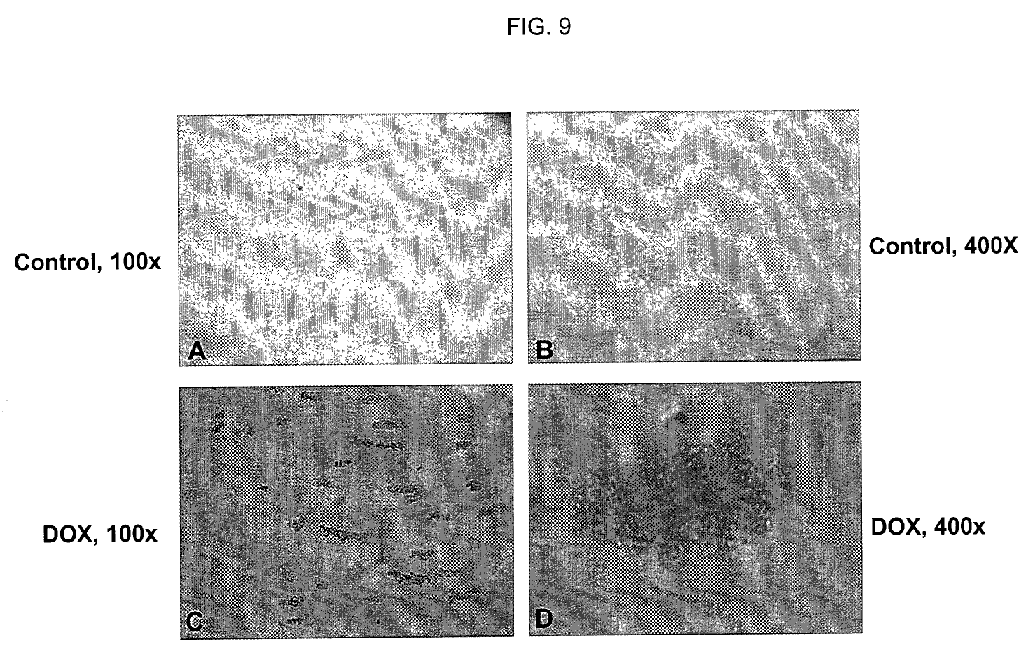

HSAM nanostructures derived from 4×10¹³ molecules of HSAM signal probe (SEQ ID No.1) and 3×10¹³ molecules of avidin were incubated with 1 μl of doxorubicin (2.5 μg) at room temperature for 30 min. The mixture was spun at 8,000 rpm for 3 min. The pellet was re-suspended in 10 μl of Tris Buffer (same as in Example 1), applied onto a glass slide and examined under a microscope. In the control, the HSAM nanostructures were treated in the same manner as mentioned above with exception that 1 μl of vehicle, instead of doxorubicin, was added to the same amount of HSAM nanostructures. The results are depicted in FIG. 9. Doxorubicin is red in color and can be visualized microscopically when bound to the HSAM nanostructure. In the absence of doxorubicin, the HSAM particle was clear as depicted in Panels A and B at 100× and 400× magnification, respectively. Once doxorubicin bound to the HSAM nanostructure, the nanostructure turned dark (i.e., red), depicted in Panels C and D at 100× and 400× magnification, respectively, indicating that doxorubicin was able to bind to HSAM particle at a high concentration.

### Example 6

Binding Capacity of Doxorubicin to HSAM Nanostructure

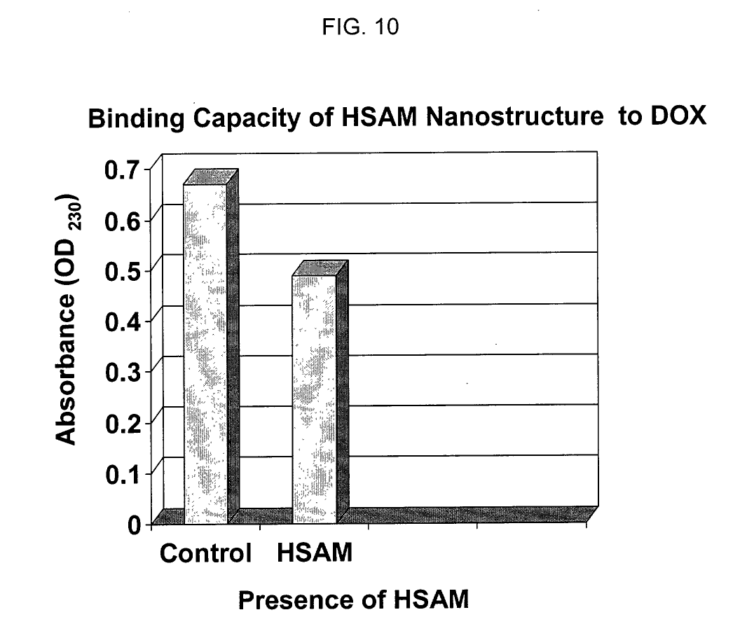

To determine the binding capacity of doxorubicin to HSAM nanostructures, 1 μl doxorubicin (2.5 μg) and 10 μl HSAM nanostructures were mixed and incubated at room temperature for one hour with shaking. The mixtures were spun at 8,000 rpm for two minutes. In the control, 1 μl of doxorubicin was added to 10 μl of buffer. 1 μl of supernatant from each tube was added to 49 μl of H₂O. The absorbance at OD₂₃₀ was measured and the results are shown in FIG. 10. The difference from the control and HSAM represents a 27% decrease in the OD₂₃₀ value, which indicated that 1.6×10¹³ molecules of HSAM nanostructures, equivalent to 1.5 μg of HSAM signal probe, can bind to 1 μg doxorubicin, indicating a high binding capacity between doxorubicin and the HSAM nanostructures.

### Example 7

Inhibition of Gastric Adenocarcinoma Cell Growth by HSAM Nanostructures that Incorporate Doxorubicin

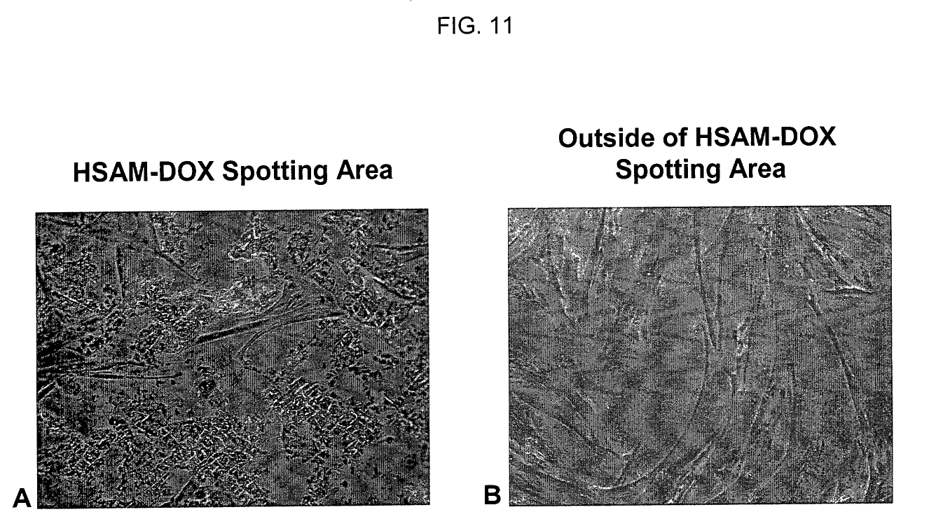

The HSAM—doxorubicin (HSAM-DOX) nanostructure was prepared in the same manner previously described in Example 6. A 5 μl aliquot of the nanostructure was spotted onto a cell culture dish and allowed to air-dry in order to allow the HSAM-DOX nanostructure to bind on the surface. Gastric carcinoma cells (patient sample) were inoculated onto the dish in the presence of appropriate medium and the culture was incubated in a CO₂ incubator for 3 days. The results, depicted in FIG. 11, showed that the cells did not grow adjacent to the HSAM-DOX nanostructure, indicating that doxorubicin was able to be released and inhibited cell growth. Away from HSAM-DOX nanostructure, the cells grew well, indicating that the doxorubicin was released into the microenvironment only and had little effect on distal sites.

We claim:

1. A nanostructure comprising
(a) nucleic acid molecules;
(b) one or more an affinity pairs;
(c) one or more agents; and
(d) a biocompatible matrix.

2. The nanostructure of claim 1, wherein the affinity pair consists of a ligand and a ligand binding moiety.

3. The nanostructure of claim 1, wherein the affinity pair comprises polynucelotide sequences that are complementary to one another.

4. The nanostructure of claim 1, wherein the nucleic acid molecules are selected from the group consisting of DNA and RNA.

5. The nanostructure of claim 2, wherein the ligand is selected from the group consisting of biotin, an antigen, a hapten, an antibody, a heavy metal derivative and a polynucleotide.

6. The nanostructure of claim 5, wherein the polynucleotide is selected from a group consisting of poly dC, poly dA, poly dG, poly dT, and poly U.

7. The nanostructure of claim 2, wherein the ligand binding moiety is selected from the group consisting of streptavidin, avidin, an antibody, an antigen, a thio group and a polynucleotide.

8. The nanostructure of claim 7, wherein the polynucleotide is selected from a group consisting of poly dC, poly dA, poly dG, poly dT, and poly U.

9. The nanostructure of claim 2, wherein the ligand is biotin.

10. The nanostructure of claim 2, wherein the ligand binding moiety is avidin.

11. The nanostructure of claim 1, wherein the nanostructure is administered to a subject suffering from a disease, infection or disorder.

12. The nanostructure of claim 1, wherein the nanostructure is administered to a subject in order to ascertain whether the subject suffers from a disease, infection or disorder.

13. The nanostructure of claim 1, wherein the nanostructure is administered to a subject in order to elicit an immune response.

14. The nanostructure of claim 1, wherein the agent is selected from the group consisting of a protein, a peptide, a small molecule, a chemical compound, a chemotherapeutic, a chemosensitizer, a radiosensitizer, an antibody, a lipid, a dye, a metal, an ion, a bioluminescent molecule, a chemiluminescent molecule, a fluorescent molecule, a radioactive molecule, a drug and a polynucleotide.

15. The nanostructure of claim 14, wherein the chemical compound is doxorubicin.

16. The nanostructure of claim 14, wherein the protein is insulin.

17. The nanostructure of claim 14, wherein the polynucleotide encodes for a polypeptide.

18. The nanostructure of claim 14, wherein the polynucleotide functions to prevent the expression of a target gene.

19. The nanostructure of claim 18, wherein the polynucleotide is selected from the group consisting of a RNAi molecule, an antisense molecule and a ribozyme molecule.

20. The nanostructure of claim 14, wherein the polynucleotide is modified.

21. The nanostructure of claim 1, wherein the nanostructure further comprises a pharmaceutically acceptable carrier.
